(19) United States
(12) Patent Application Publication
(10) Pub. No.: US 2001/0036283 A1
(43) Pub. Date: Nov. 1, 2001

(54) Active noise reduction system

(76) Inventor: Mark Donaldson, Alfriston (NZ)

Correspondence Address: YOUNG & THOMPSON, 745 SOUTH 23RD STREET 2ND FLOOR, ARLINGTON, VA, 22202

(21) Appl. No.: 09/799,714

(22) Filed: Mar. 7, 2001

(30) Foreign Application Priority Data
Mar. 7, 2000 (NZ) 503221
Mar. 8, 2000 (NZ) 503242

(51) Int. Cl. A61F 11/06; G10K 11/16; H03B 29/00

(52) U.S. Cl. 381/71.11; 381/71.8; 381/71.1; 381/94.1

(57) ABSTRACT

An active noise reduction system is provided having a novel configuration that uses a fixed point digital filter to estimate an inverted replica of the acoustic noise from the measurement of the control error at some predefined position. The inverted replica of the measured acoustic noise is used to generate an accurate acoustic control response that is processed by a fixed point digital filter in order to compensate for the undesirable dynamic effect of the physical components comprising the system. The system in effect yields a configuration that is open loop and which can provide an acoustic control response with an ability to generate a close match of the inverted replica of the acoustic noise. The system is not constrained by closed loop stability concerns which occur when employing an analogue feedback compensation approach. Nor is the configuration of the present invention hindered by poor parameter convergence as in the case of an adaptive feedforward implementation.

### FIELD OF INVENTION

This invention relates to active noise reduction systems.

### BACKGROUND OF THE INVENTION

Formulating practical solutions for the reduction of problematic noise is an active area of engineering research in both the fields of acoustics and control. To date, noise reduction has been mostly carried out using passive means. These passive methods almost always require the installation of heavy, bulky and costly materials such as foams, wools and fibrous bats. The additional weight bulk and physical change required is in many situations neither practicable nor cost effective.

Also, one of the fundamental problems with insulators or absorbing materials is that they do not work well at reducing noise at the low frequencies. This is primarily because the acoustic wavelength at low frequencies becomes large compared to the thickness of typical absorbent materials.

Active noise reduction can overcome these problems and disadvantages. Active noise reduction is based on the principle of superposition of signals. According to the principle of superposition, if two signals exist, one an undesired disturbance, the other a controlled response, their combined effect can be made zero if they are equal in magnitude and opposite in phase. This signal cancellation phenomenon is commonly termed destructive interference, and is a basis for the operation of active noise reduction systems.

The advantages of active noise reduction are numerous. However, the two most significant relate to the method's spectral effectiveness and method of installation.

Active noise reduction exploits the long wavelengths associated with low frequency sound. Active noise reduction systems are, therefore, more effective at attenuating low frequency acoustic disturbances. Such low frequency disturbances are the common undesired side effect of operating machinery and are difficult to reduce using passive techniques.

In terms of physical implementation, active noise reduction systems typically comprise small and light weight components. This means that active noise reduction systems can be used in many situations where passive methods are impractical due to their bulk, weight and cost effectiveness.

The existing active noise reduction systems still suffer from their own disadvantages, however. These include the risks associated with system stability, less than adequate noise suppression performance and insufficient operating bandwidth.

Active noise reduction systems based on a feedback control approach, for example, risk instability, particularly where the feedback compensator has no means of accounting for change in the dynamic characteristics of the plant. It is difficult to design a feedback compensation network that provides both highly effective and robust noise reduction, particularly over a wide frequency bandwidth. Also, as the feedback compensator's gain is increased to improve low frequency noise suppression, amplification at the higher frequencies typically impacts negatively on performance.

Active noise reduction systems based on the known adaptive feedforward techniques, for example, can experience problems with effective parameter convergence and therefore provide less than optimal performance. Adaptive techniques also require intensive processing particularly where the feedforward path dynamics are complex and the time available to compute a control response is brief. In many cases this makes this method of control unfeasible due to cost or the inability to implement the system practically.

### OBJECT

It is an object of the present invention to provide an improved active noise reduction system or to at least to provide the public with a useful choice.

### SUMMARY OF THE INVENTION

In one aspect the invention may broadly be said to consist in an active noise reduction apparatus including:

a sound source means provided in a sound field,

a sensing means provided in the sound field for providing an input signal corresponding to sound from the sound source means and noise in the sound field,

a processing means including

a noise signal estimation means for producing a noise estimate being an estimate of a component of the input signal corresponding to the noise, and

an inversion means for processing the noise estimate to produce an output signal which is used to drive the sound source means, and whereby

the sound source means provides sound in the sound field which is of substantially equal amplitude and opposite phase to the noise in the sound field thereby substantially reducing the noise by destructive interference.

Preferably the noise signal estimation means includes a model of the open loop dynamics of the apparatus and the output signal is applied to the model to provide an estimate of the input signal which is substantially devoid of the noise component.

Preferably the apparatus further includes algebraic adding means to add the estimated input signal which is substantially devoid of the noise component to the input signal to derive an estimate of the noise component.

In a further aspect the invention may broadly be said to consist in an active noise reducing control method, the method comprising the steps of sensing sound in a sound field, the sound including sound produced from a sound source means provided in the sound field, and noise in the sound field,

providing at least an estimated noise component being an estimate of a component of the sensed sound corresponding to the noise,

applying the estimated noise component to a model of an inversion of the open loop system dynamics to produce a driving signal to the sound source means.

In a still further aspect the invention may broadly be said to consist in an active noise reduction system having a sensing means to sense sound produced by a sound source in a sound field, and noise in the noise field,

the sensed signal being provided to a fixed point digital filter to estimate an inverted replica of the sensed noise in a noise field

the inverted replica of the sensed noise being provided to a second fixed point digital filter having means to compensate for the undesirable dynamic effect of the physical components comprising the system, and

the output of the second digital filter being provided to the sound source whereby the sound source unit processes the signal to produce sound in the sound field which substantially destructively interferes with the noise in the sound field.

In a further aspect the invention may broadly be said to consist in an open loop active noise reduction system according to any one of the preceding statements of invention.

In a further aspect the invention may broadly be said to consist in a feedforward control method for an active noise reduction system according to any one of the preceding statements of invention.

In another aspect, the invention resides in an active noise reduction system that utilises a digital filter to obtain a signal indicative of the noise desired to be reduced by the system, and to invert the noise signal to formulate a controlling acoustic response which when combined with the acoustic noise at a position of control error measurement results in a substantial cancellation of both signals via the mechanism of the destructive interference of signals.

The fixed point digital filter outputs to an acoustic actuator a compensated estimate of the inverted acoustic noise signal present at a measurement and control position. The compensation effected is an accurate and stable inversion of the active noise reduction system's open-loop dynamics, that is, the dynamics of the combined system components located between the output and input terminals of the active noise reduction electronic circuitry.

The active noise reduction system preferably comprises one or more acoustic actuator(s), active noise reduction electronic circuitry required to physically implement the fixed point digital filter, and one or more acoustic sensor(s).

The digital component of the active noise reduction electronic circuitry preferably comprises one or more digital-signal-processors (DST), one or more analogue-to-digital (ADC) converters and one-or more digital-to-analogue converters (DAC).

The analogue component of the active noise reduction electronic circuitry preferably comprises on the input side a preamplifier and on the output side a power amplifier.

Preferably the digital sampling frequency selected is high enough such that the level of acoustic signal present at frequencies equal to or greater than the Nyquist frequency falls well below the noise floor of the analogue-to-digital converter so as to eliminate any need for anti-aliasing filtering.

Preferably the digital sampling frequency selected is high enough to eliminate any need for reconstruction filtering.

Preferably the analogue-to-digital converters and digital-to-analogue converters used at the input and output of the digital-signal-processor respectively exhibit a very low group delay.

Preferably the DSP, ADC and DAC devices are embodied in one piece of silicon known as a mixed-mode application-specific-integrated-circuit (ASIC) to minimise processing latency, reduce the phase-lag gradient and improve noise reduction performance.

Preferably a distance separating the acoustic actuator and sensor is set as low as possible to reduce the phase-lag gradient of the open-loop system and improve noise reduction performance. More preferably the distance between the acoustic actuator and acoustic sensor is zero.

In another version of the invention, a simple analogue feedback compensator augments the DSP, deriving signal from the acoustic sensor and outputting to the acoustic actuator and to the DSP via an ADC to yield a hybrid digital-analogue active noise reduction implementation.

Preferably the analogue feedback compensatory dynamics are designed to cancel any remaining low frequency noise. This is preferably achieved by employing an analogue controller comprising a cascaded network of phase-lag and/or low pass filters.

In another version of the invention, a programme audio reference is input to the DSP via an ADC and is output as part of the acoustic control response. This reference signal is not cancelled during any noise cancellation.

### BRIEF DESCRIPTION OF THE DRAWINGS

In order that this invention may be more readily understood and put into practical effect, reference will flow be made to the accompanying drawings which illustrate one or more preferred embodiments of the invention and wherein:

FIG. 1 is a schematic of the configuration of components comprising the system of the invention.

FIG. 2 is a block diagram of the system of FIG. 1.

FIG. 3 is a diagram of a practical implementation of the system of FIG. 1.

FIG. 4 is a schematic of the system of FIG. 1 but with a programme audio reference included.

FIG. 5 is a block diagram of the system of FIG. 4.

FIG. 6 is a diagram of a practical implementation of the system of FIG. 4.

FIG. 7 is a schematic of the system of FIG. 1 but with a programme audio reference and analogue feedback compensator included.

FIG. 8 is a block diagram of the system of FIG. 7.

FIG. 9 is a diagram of a practical implementation of the system of FIG. 7.

FIG. 10 is an illustration of the invention embodied as an active headset device providing noise cancellation within the ear piece.

FIG. 11 is an illustration of the invention embodied as an active panel device providing cancellation near and around the panel.

FIG. 12 is a perspective view of further active panel device according to the invention.

### DETAILED DESCRIPTION OF THE INVENTION

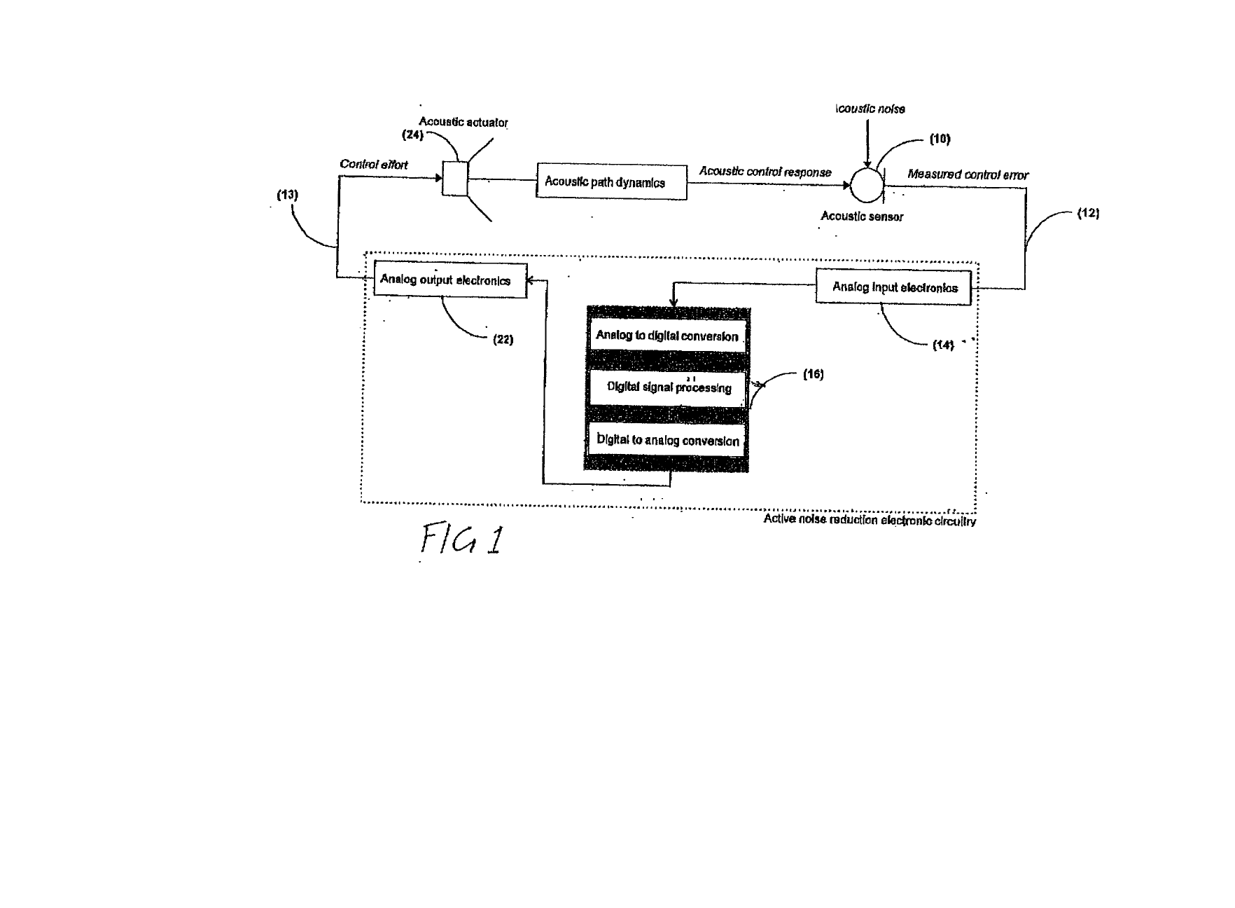

Reference is now made to FIGS. 1 to 9 where a schematic and several block diagrams of active noise reduction systems are shown.

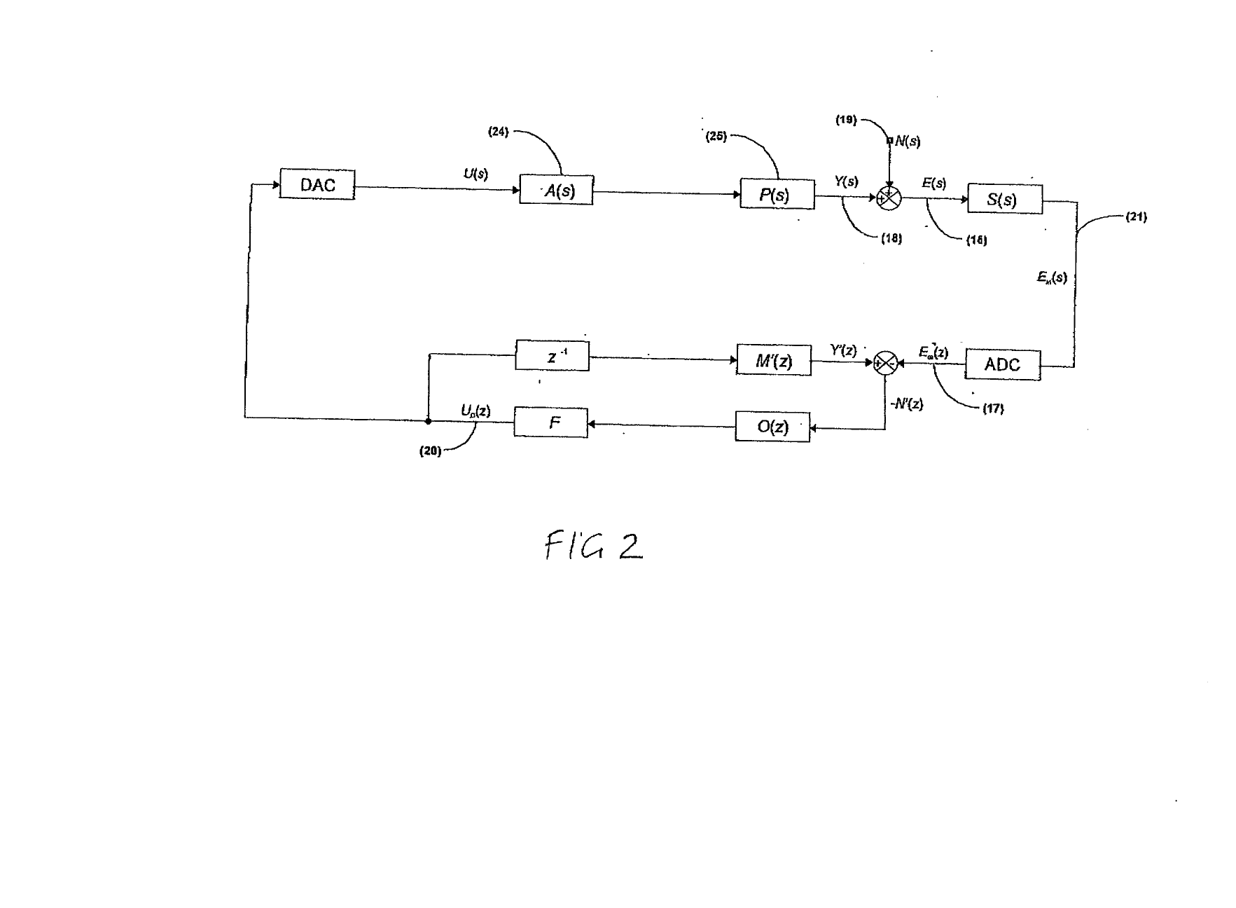

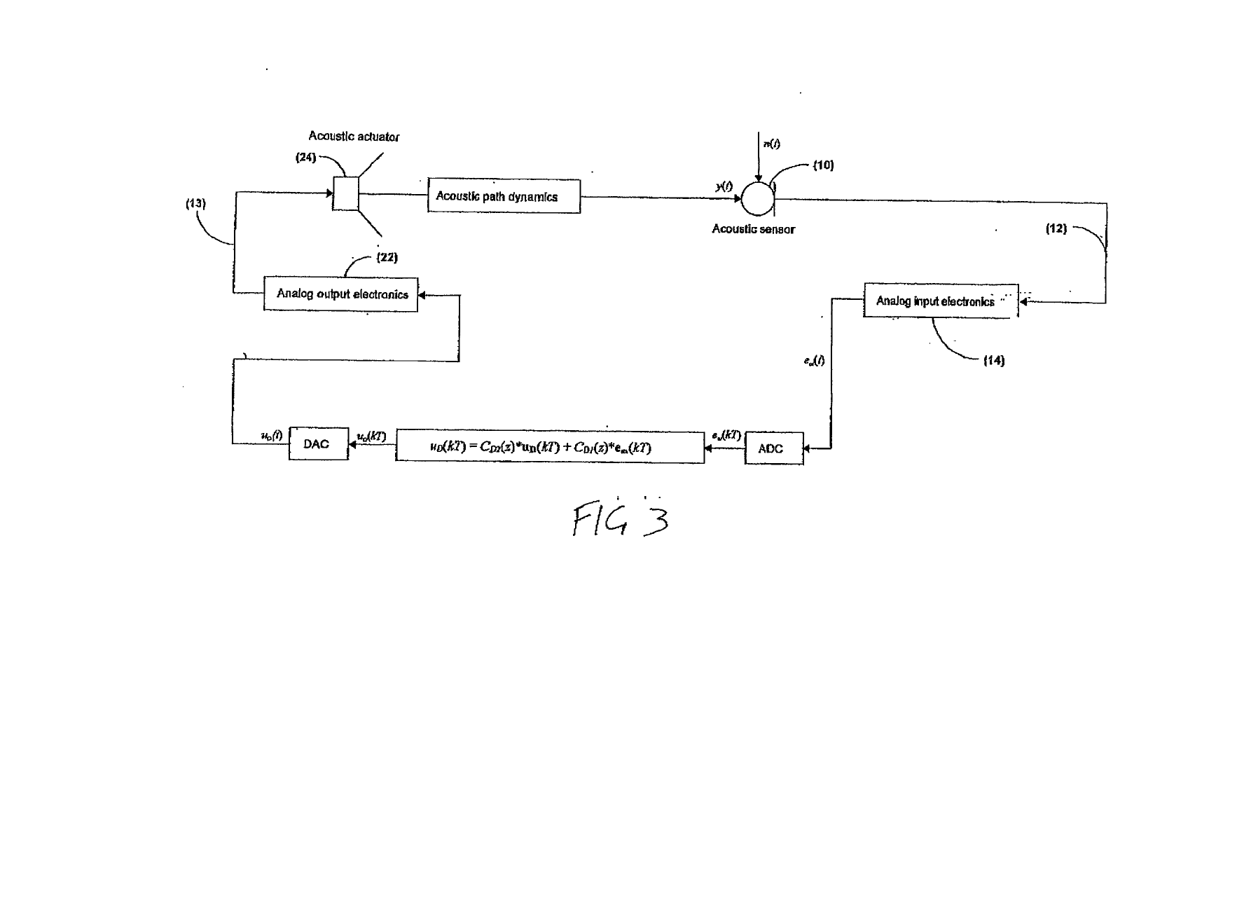

The components of the schematic diagrams i.e. those of FIGS. 1, 4 and 7 are represented in the block diagrams i.e. FIGS. 2, 5 and 8 by their mathematical denotations in the complex frequency domain. Mathematical relationships relevant to operation of the active filters of the systems shown in the diagrams are also shown in the diagrams of examples of practical implementations of the systems in FIGS. 3, 6 and 9.

In the schematic diagrams the acoustic sensor (10) with associated components such as cables and connectors (12) is represented as block S(s) in the block diagrams.

The active noise reduction electronics shown in the schematic diagrams incorporates the analogue input electronics (14), the digital-signal-processor and the analogue-to-digital and digital-to-analogue converters (16), and the analogue output electronics (22).

In the schematic diagrams, the acoustic actuator (24), with associated components such as cables and connectors (13), is shown as block A(s) in the block diagrams.

A digital filter, preferably a fixed point filter, implemented physically on DSP, determines an appropriate control effort, u_{D}(kT) (20) (designated U_{D}(z) in the block diagrams) based on the measured and sampled control error signal, eₘ(kT), (17) (designated Eₘ(z) in the block diagrams) according to the following control law,

$$\frac{{U}_{D} ( z )}{{E}_{m} ( z )} = \frac{{C}_{D1} ( z )}{1 - {C}_{DZ} ( z )} \qquad \text{(1a)}$$

u_{D}(kT)=C_{DZ}(z)*u_{D}(kT)+C_{D1}(z)*eₘ(kT) (1b)

where C_{D1}(z) and C_{D2}(z) represent the filter parameters in the complex frequency domain, u_{D}(kT) represents the vector of n current and past values of control effort according to {u_{D}(kT), u_{D}(k-1)T), u_{D}(k-2)T) . . . u_{D}(k-n)T)}, eₘ(kT) represents the vector of m current and past values of measured and sampled error according to {eₘ(kT), eₘ((k-1)T), eₘ((k-2)T) . . . eₘ(k-m)T)}, C_{D1}(z) and m denotes the number of order of C_{D1}(z).

The design of the filter terms, C_{D1}(z) and C_{D2}(z), is based on the following:

The control error, e(t), is the summation of the acoustic control response, y(t), (18 and designated Y(s) in the block diagrams) and the acoustic noise, n(t), (19 and designated N(s) in the block diagrams), at the predefined position of control and measurement, or,

e(t)=y(t)+n(t) (2)

The measured control error, eₘ(t), (21 and designated Eₘ(s) in the block diagrams) is the control error, e(t), (16 and designated as E(s) in the block diagrams), processed by the acoustic sensor, S(s) according to,

Eₘ(s)=S(s)E(s) (3)

Furthermore from equations 2 and 3 the measured and sampled control error can be acquired according to,

eₘ(kT)=Yₘ(kT)+nₘ(kT) (4)

where Yₘ(kT) denotes the sampled measured acoustic control response, and nₘ(kT) denotes the sampled measured acoustic noise. Both Yₘ(kT) and nₘ(kT) can not be directly measured.

To provide maximum cancellation at the position of control the acoustic control response, y(t), when reaching this position, must closely match the inversion of the acoustic noise, or −n(t). For the sampled data stream, therefore, Yₘ(kT) must closely match −nₘ(kT).

As the acoustic noise can be directly measured it is estimated according to,

n′ₘ(kT)=eₘ(kT)−z⁻¹M′(z)* u_{D}(kT) (5)

where M′(z) represents a discrete time model of the open loop dynamics of the combined system components of the plant, or,

M(s)=S(s)A(s)P(s) (6)

where A(s) (24 in the block diagrams) and P(s) (25 in the block diagrams) represent the dynamics of the acoustic actuator and acoustic path respectively.

Preferably, M′(z) is determined using accurate spectral analysis. For example, a high resolution frequency-response-function of the system between the input to A(s) and the output of S(s) can be measured. An inverse Fourier transform of this complex data will yield an accurate finite-impulse response (FIR) filter representation of M(s).

After acquiring an accurate estimate of the inverse of the acoustic noise, −nₘ′(kT), this signal is processed by a filter FO(z), representing an accurate and stable inverse of M(s), in terms of both phase and magnitude, according to,

U_{D}(z)=FO(z).−N′(z) (7)

in order to compensate for the dynamic effect of the system components. These components alter the phase and magnitude of the signal, −nₘ′(kT), directly or indirectly during its estimation, actuation and transmission. F denotes a scalar gain term introduced to provide a means of adjusting the gain of the control effort, u_{D}(kT).

When M′(z) is obtained in FIR form preferably O(z) is calculated by employing optimal or robust signal processing techniques. For example, M′(z) maybe transformed into an equivalent state-variable representation where an optimal and fully recursive filter, O(z), maybe determined by using linear-quadratic-regulator (LQR) design techniques.

By substituting equation 5 into equation 7, the control law,

U_{D}(z)=−F.O(z)Eₘ(z)+z⁻¹F.O(z)M′(z)U_{D}(z) (8a)

or in the time domain,

u_{D}(kT)=−F.O(z)*eₘ(kT)+z⁻¹F.O(z)M′(z)*u_{D}(kT) (8b)

is obtained.

By defining for the purpose of simplification,

C_{D1}(z)=−F.O(z) (9a)

and

C_{D2}(z)=z⁻¹F.O(z)M′(z) (9b)

equation 1 is yielded.

This equation is implemented physically in the time domain by using a DSP device of sufficient power to process this filter at the selected sampling frequency 1/T. The sampling frequency selected is high enough such that the level of acoustic signal present at frequencies equal to or greater than the Nyquist frequency falls well below the noise floor of the analogue-to-digital converter so as to eliminate any need for anti-aliasing filtering.

Also, the sampling frequency selected is high enough to eliminate any need for reconstruction filtering.

The DSP has as its input the measured and sampled control error, eₘ(kT), that is provided by an ADC device. The ADC is connected, via auxiliary analogue electronics and associated cabling (12), to the acoustic sensor (10). The digital fixed point filter processed in the DSP outputs a stream of control effort values, u_{D}(kT), to a DAC device where it is transformed, into an analogue continuous signal and then transmitted to the acoustic actuator (24) via some auxiliary analogue electronics (22) and associated cabling (13). The control effort is converted into an acoustic response and it then passes to the measurement position (10) via the acoustic path where on arrival it is termed the acoustic control response and ideally combines with the acoustic noise to provide significant acoustic noise reduction. In practice, the DSP′, ADC and DAC devices are embodied in one piece of silicon known as a mixed-mode application-specific-integrated-circuit (ASIC) to minimise processing latency, reduce the phase-lag gradient and improve noise reduction performance.

The filter parameters, C_{D1}(z) and C_{D2}(z) are preferably stored on a memory device within the active noise reduction system's electronic circuitry. These parameters would be loaded to the DSP device on booting. Alternatively they maybe stored external to the electronic circuitry but downloaded to it by a cable or other electronic means.

Referrng to FIG. 3, the system of FIG. 1, together with the mathematical model of the active filter required to implement that system is shown.

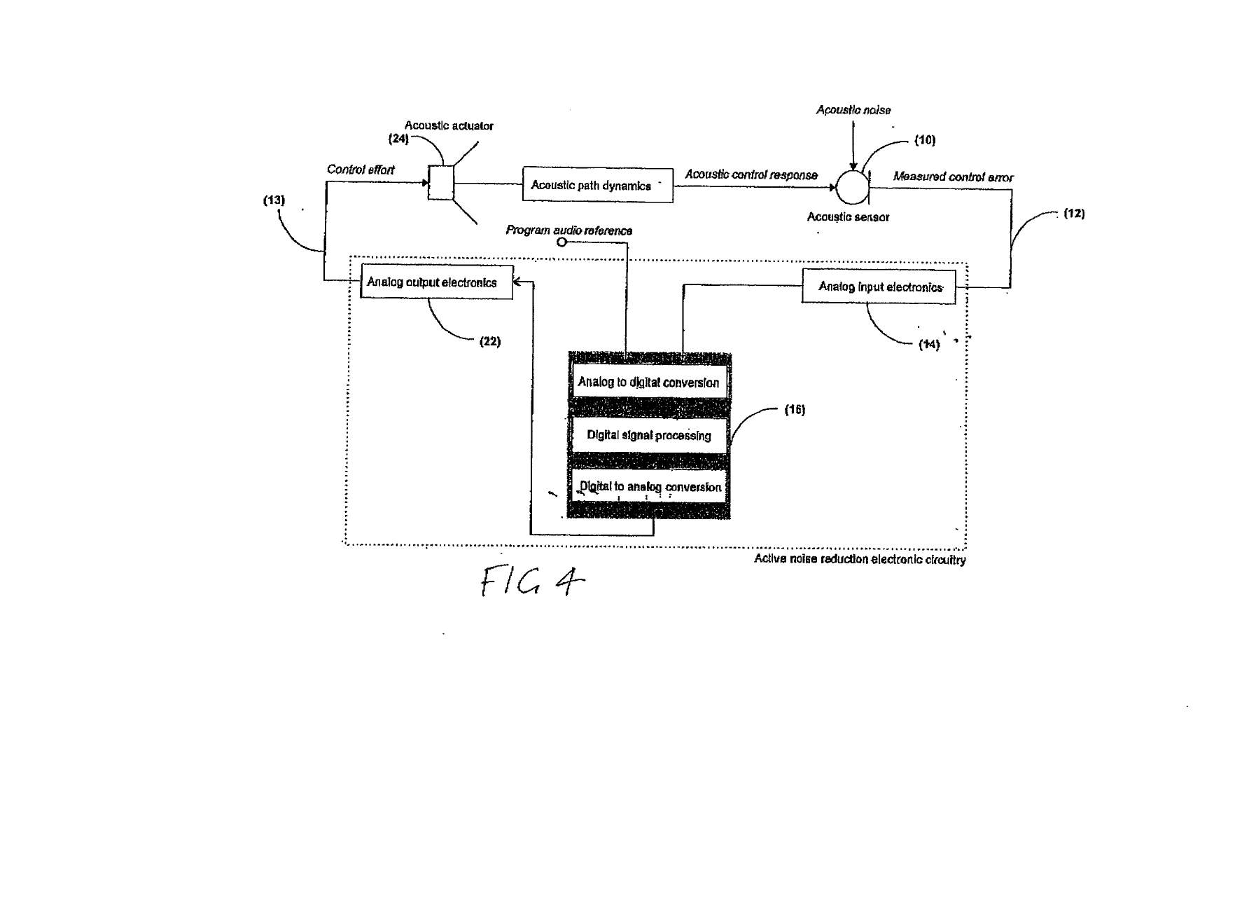

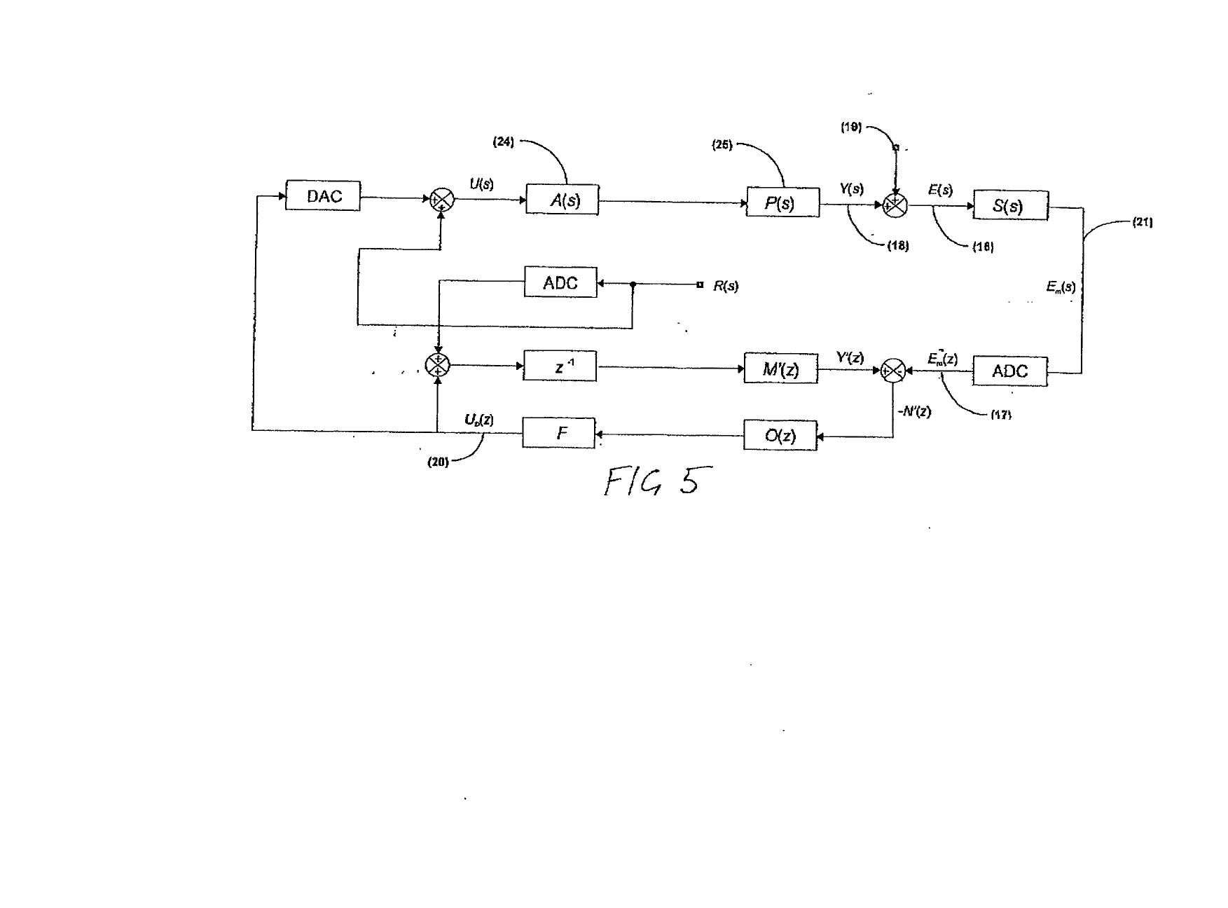

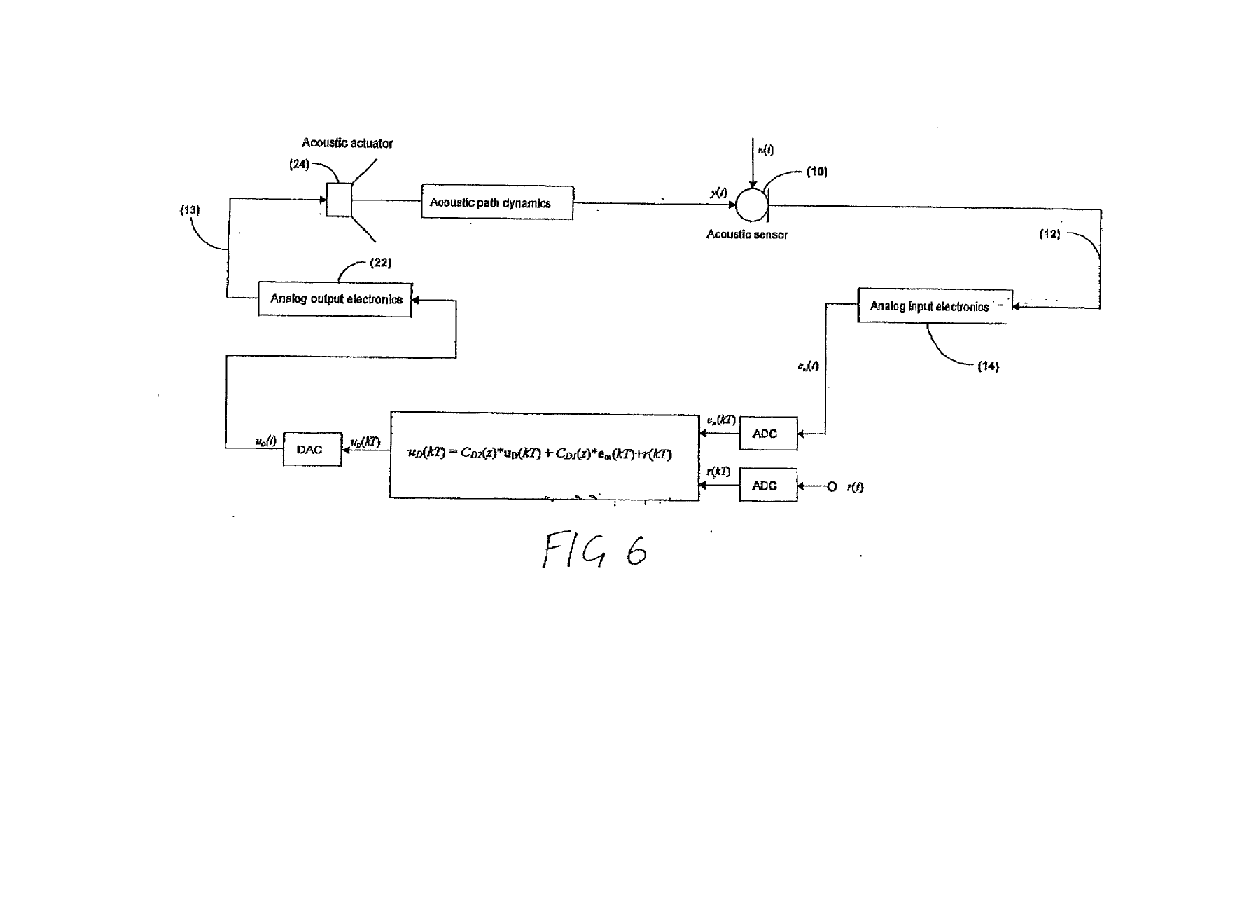

In FIG. 4, the schematic shows provision of an analogue program audio reference to the system. The analogue reference signal is processed by the processing section (16) so as to be provided as an audio signal to the actuator (24) together with the necessary signal to provided noise cancellation at the sensor (10). In FIG. 5 the reference signal, represented as R(s) is added to the analogue driving signal provided to the actuator (24). R(s) is also processed to provided a digital signal which is added to the digital control effort for provision to the open loop plant estimation and is thus compensated for by the system so that the correct inversion of the estimated noise is provided to the optimal inversion filter. In FIG. 6 a practical implementation is illustrated showing the reference signal in digital form, r(kT), being added to the control effort to thereby be provided to the acoustic path or sound field. Therefore, a reference signal corresponding to sounds such as music may be provided to the acoustic path and will appear to a listener in the vicinity of the sensor (10) to be substantially free of background noise. The reference signal could also correspond to a signal from a public address system for example.

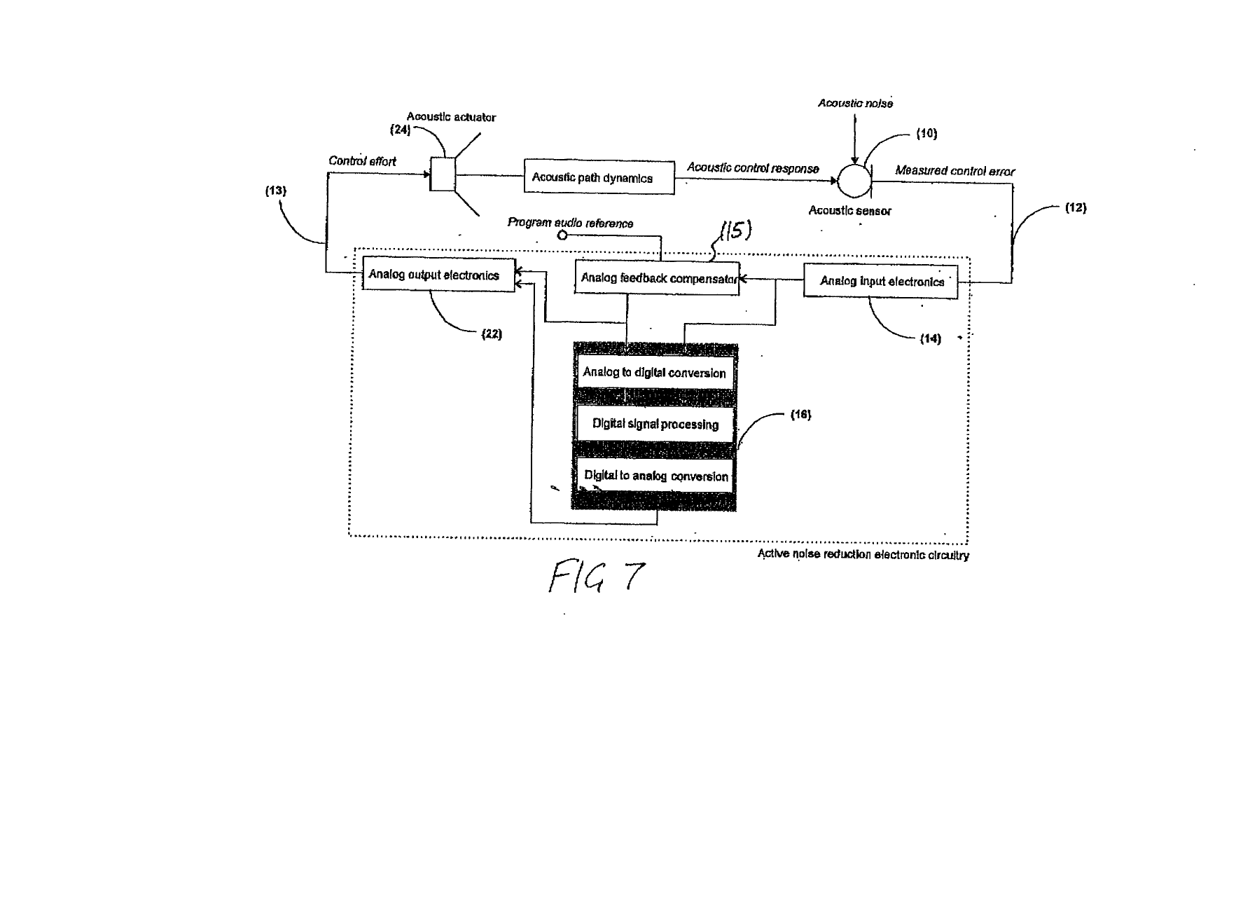

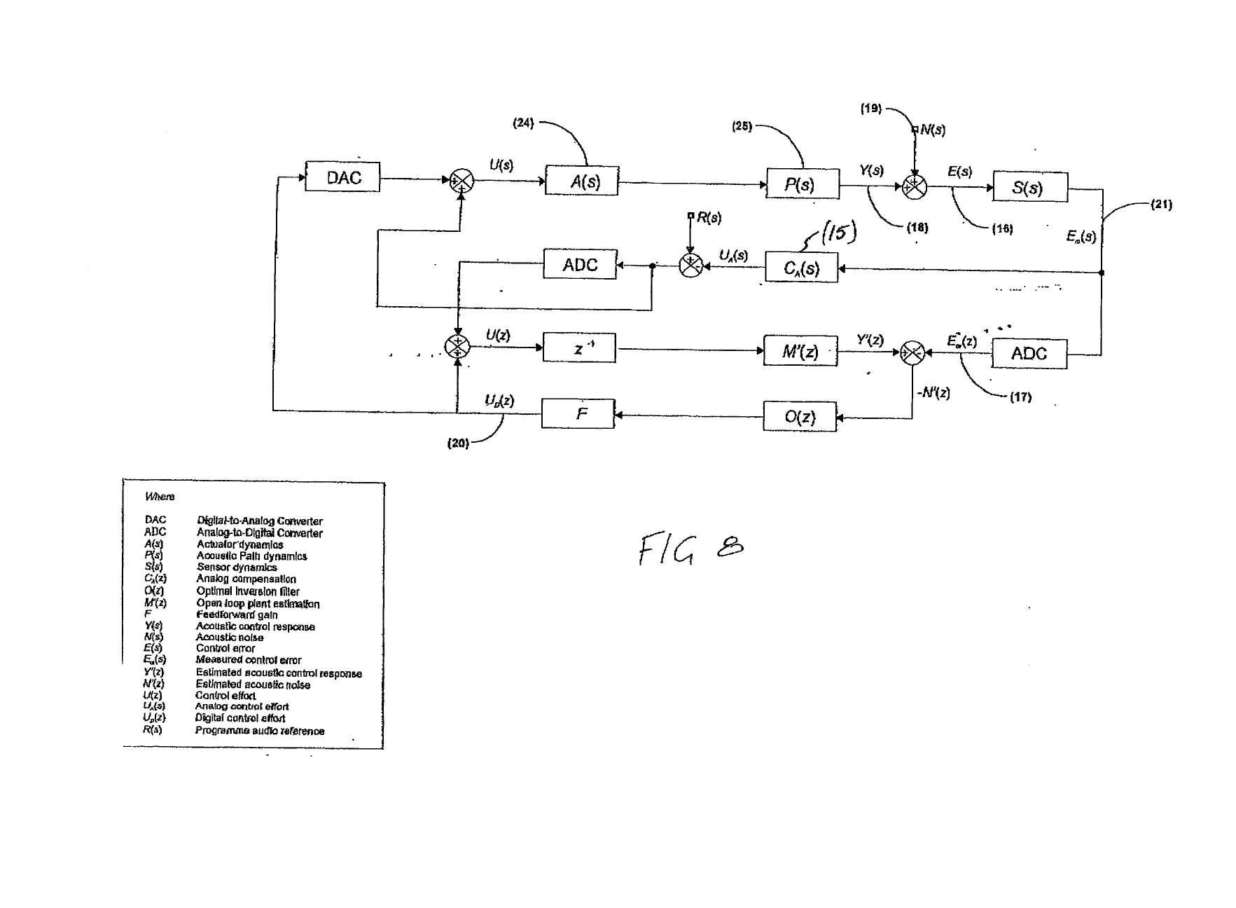

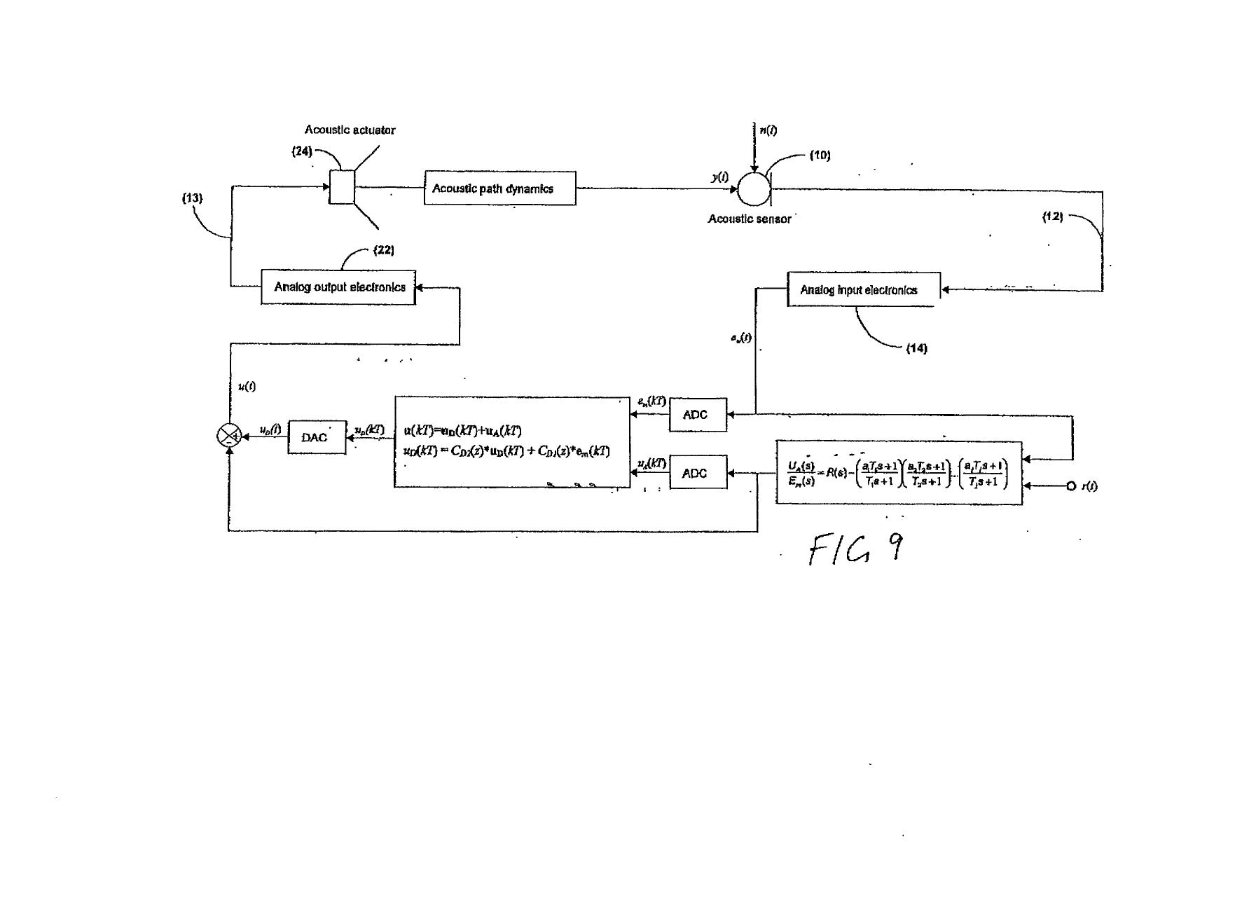

Referring to FIG. 7, the program audio reference signal is shown provided to an analogue feedback compensator (15) which augments the digital signal processor to yield a hybrid digital-analogue active noise reduction implementation. The analogue feedback compensatory dynamics are designed to cancel any remaining low frequency noise. In the practical implementation of FIG. 9, it will be seen that the compensation is achieved by a cascaded network of phase-lag or low pass filters. Turning to FIG. 8, the block diagram shows the analogue control effort produced by the analogue feedback compensator (15) being subtracted from the reference signal and the result added to the analogue output of the digital control effort. The digital processing circuitry compensates for this by adding a digital form of the analogue control effort to the digital control effort provided to the open loop plant estimation to thereby provide a compensated inverted noise estimation.

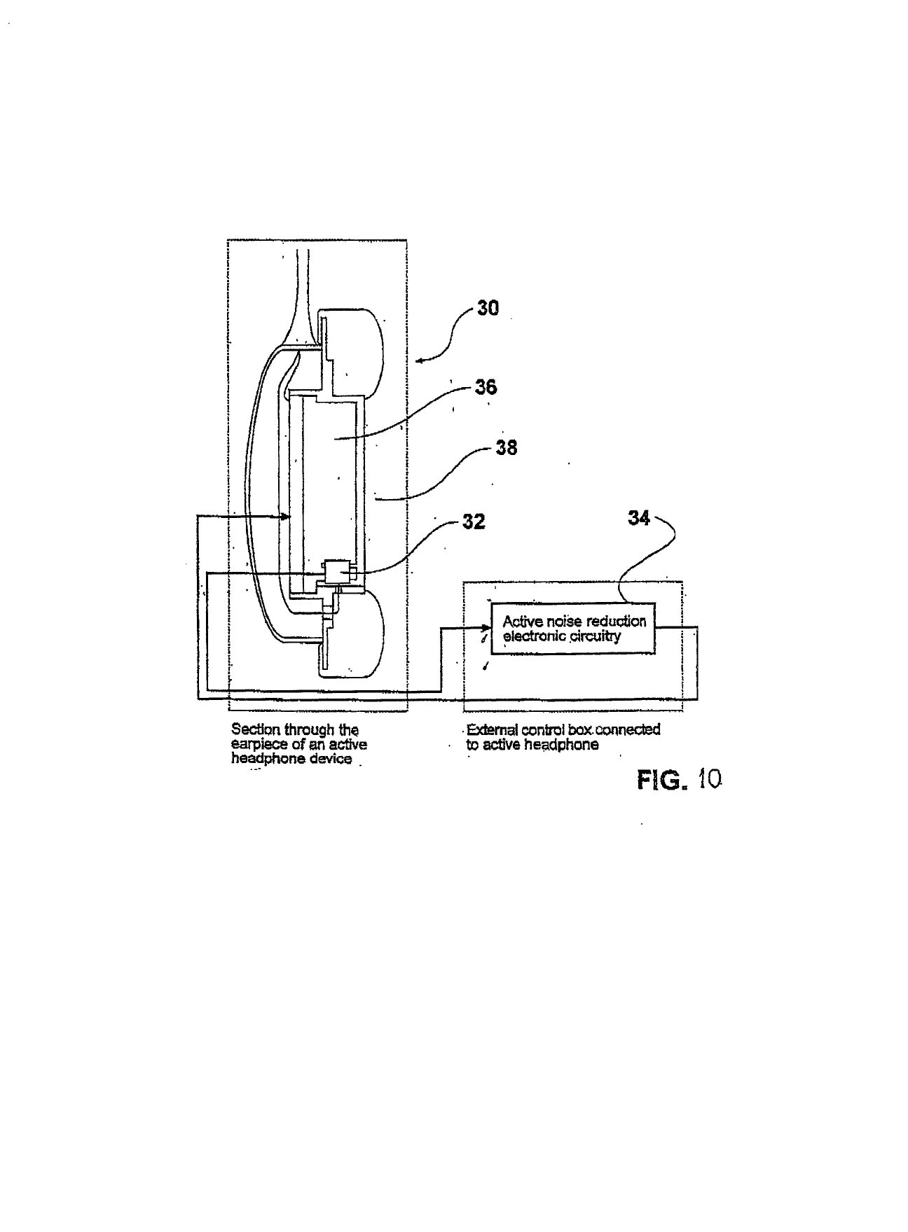

In FIG. 10 the system is embodied as an active headset (30). The acoustic sensor (32) used here is an electret-condenser microphone (ECM). The microphone detects the control error at the measurement position and passes this to the active noise reduction system's electronic circuitry (34). Here the control effort is computed according to the developed control law and is acoustically output via a mylar speaker actuator (36). The acoustic control response and noise signals combine providing active noise cancellation within the region bounded by the earpiece (38) of the headset device and the wearer's ear (not shown).

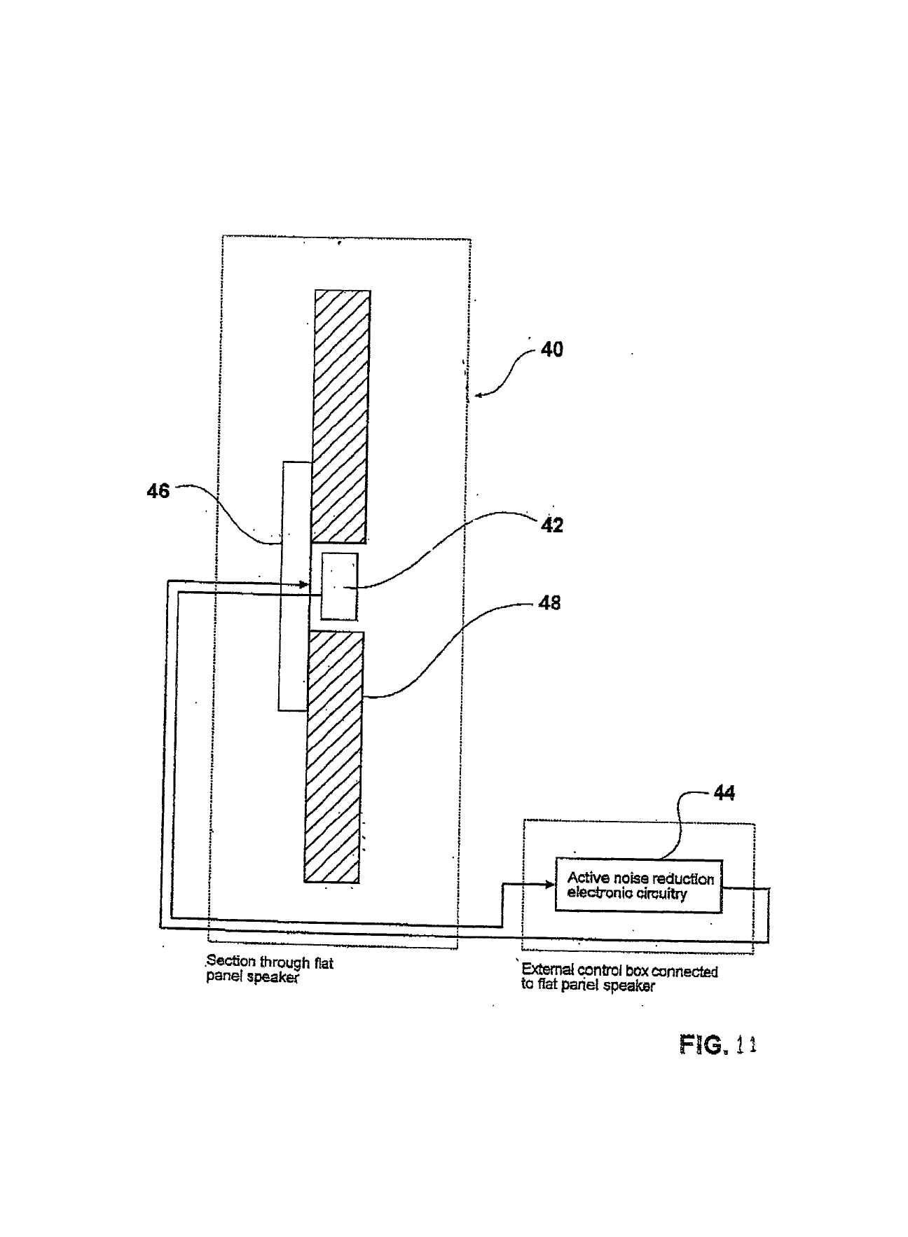

In FIG. 11 the system is embodied as an active panel loudspeaker system (40). The acoustic sensor (42) used here is an electret-condenser microphone (ECM). The microphone detects the control error at the measurement position and passes this to the active noise reduction system's electronic circuitry (44). Here the control effort is computed according to the developed control law. It is then acoustically output via an electromechanical transducer (46) to the flat panel diaphragm (48). The acoustic control response and noise signals combine providing active noise cancellation in a zone near the measurement position.

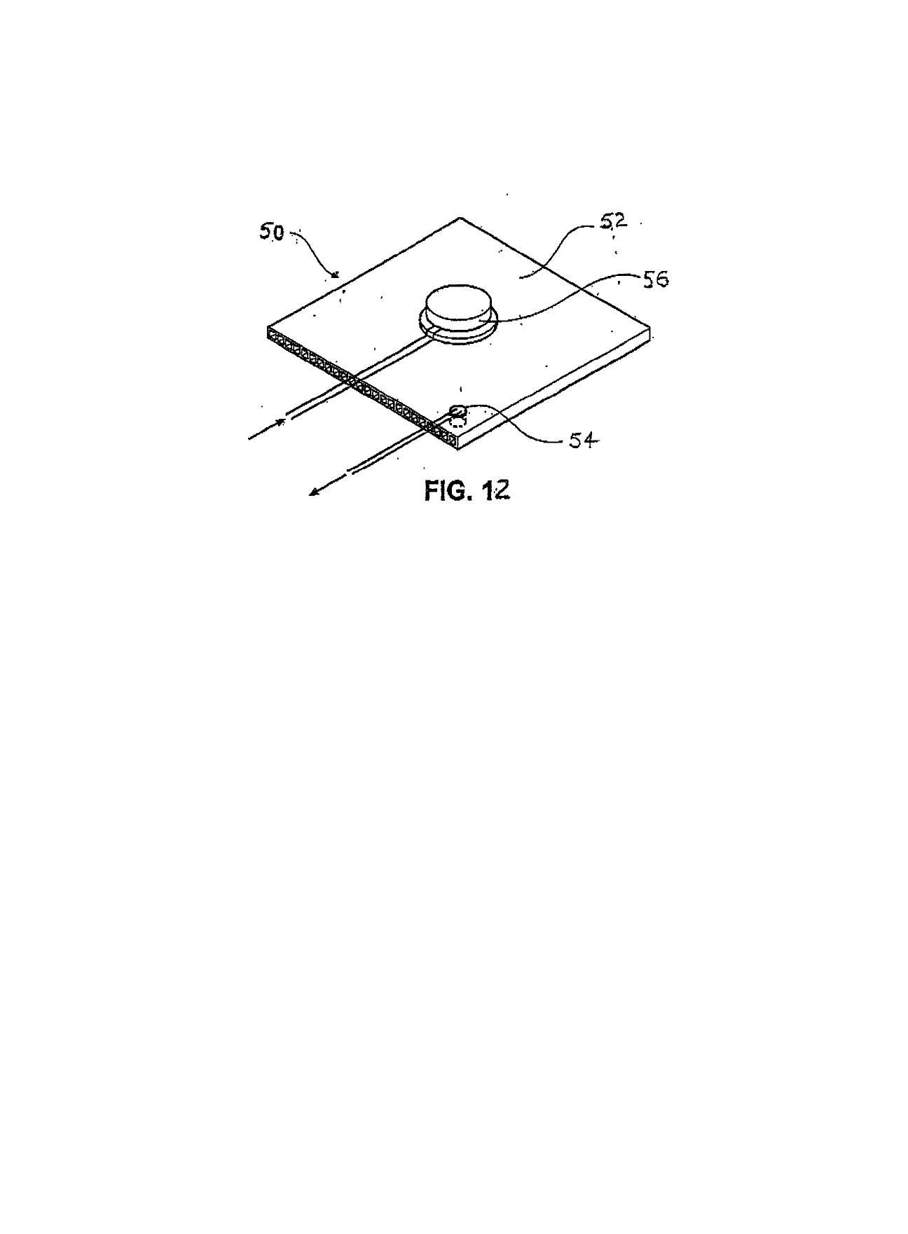

Referring now to FIG. 12 as shown a further flat or planar loudspeaker (50) incorporating noise cancellation apparatus according to one or more of the examples discussed above. The planar loudspeaker (50) has a diaphragm (52) on which there is located a microphone (54) which detects ambient noise. Ambient noise detected by the microphone (54) is sent to the noise cancelling circuitry (now shown). The noise cancelling circuitry then produces a cancellation signal as discussed above, which is then sent to the transducer (56) which causes the speaker panel and diaphragm to vibrate, thereby producing sound. The acoustic control response and noise signals combine providing active noise reduction in a zone in the vicinity of the loudspeaker.

It will be seen that a speaker of this type may be used in a variety of applications and asserted to being provided in the walls of rooms, or in parts of seat head rests, telephone phone booths or the like where it may be highly desirable to have a zone of silence. The dimensions of such a speaker and the relatively small size of the circuitry for noise suppression as set forth above create a highly desirable compact system which therefore has significant advantages over relatively more bulk and complex prior art constructions.

### VARIATIONS

It will be appreciated that various other alterations and modifications may be made to the foregoing without departing from the scope of this invention as set forth in the appended claims.

Throughout the description and claims of this specification the word “comprise” and variations of that word, such as “comprises” and “comprising”, are, not intended to exclude other additives, components, integers or steps.

1. An active noise reduction apparatus including:
a sound source means provided in a sound field,
a sensing means provided in the sound field for providing an input signal corresponding to sound from the sound source means and noise in the sound field, p1 a processing means including
a noise signal estimation means for producing a noise estimate being an estimate of a component of the input signal corresponding to the noise, and
an inversion means for processing the noise estimate to produce an output signal which is used to drive the sound source means, and whereby
the sound source means provides sound in the sound field which is of substantially equal amplitude and opposite phase to the noise in the sound field thereby substantially reducing the noise by destructive interference.

2. Apparatus as claimed in claim 1 wherein the noise signal estimation means includes a model of the open loop dynamics of the apparatus ad the output signal is applied to the model to provide an estimate of the input signal which is substantially devoid of the noise component.

3. Apparatus as claimed in claim 2 further including algebraic adding means to add the estimated input signal which is substantially devoid of the noise component to the input signal to derive an estimate of the noise component.

4. Apparatus as claimed in claim 1 wherein the inversion means include a model of an inversion of the open loop dynamics of the apparatus.

5. Apparatus as claimed in any one of the preceding claims wherein the sound source means comprises one or more acoustic actuator(s), and circuitry required to drive the actuator(s).

6. Apparatus as claimed in claim 1 wherein the processing means comprises one or more digital-signal-processors, one or more analogue-to-digital converters to sample the input signal and one-or more digital-to-analogue converters to provide the output signal in analogue form.

7. Apparatus as claimed in claim 5 wherein the processing means includes a preamplifier to amplify the input signal and a power amplifier to amplify the output signal for provision to the acoustic actuator.

8. Apparatus as claimed in claim 6 wherein the digital sampling frequency of the analogue-to-digital converter is selected to be high enough such that the level of acoustic signal present at frequencies equal to or greater than the Nyquist frequency falls sufficiently far below the noise floor of the analogue-to-digital converter so as to eliminate any need for anti-aliasing filtering.

9. Apparatus as claimed in claim 1 wherein the sound source means and the sensing means are provided substantially adjacent to each other.

10. An active noise reducing control method, the method comprising the steps of sensing sound in a sound field, the sound including sound produced from a sound source means provided in the sound field, and noise in the sound field,
providing at least an estimated noise component being an estimate of a component of the sensed sound corresponding to the noise,
applying the estimated noise component to a model of an inversion of the open loop system dynamics to produce a driving signal to the sound source means.
